(19) United States
(12) Patent Application Publication
(10) Pub. No.: US 2023/0045255 A1
(43) Pub. Date: Feb. 9, 2023

(54) DEVICE FOR VENTILATION WITH REGULATED PRESSURE TRANSITION

(71) Applicant: Loewenstein Medical Technology S.A., Luxembourg (LU)

(72) Inventor: Matthias SCHWAIBOLD, Karlsruhe (DE)

(21) Appl. No.: 17/817,393

(22) Filed: Aug. 4, 2022

(30) Foreign Application Priority Data
Aug. 5, 2021 (DE) 102021120415.1

(51) Int. Cl. A61M 16/00

(52) U.S. Cl. CPC A61M 16/026; A61M 2016/0018; A61M 2230/40; A61M 2016/0033

(57) ABSTRACT

A ventilation device comprising a controllable respiratory gas source and a programmable control unit being configured to perform the following:
determining the respiratory gas flow, which is used to determine whether an inspiration or an expiration is present,
regulating the pressure for an inspiration (IPAP) and an expiration (EPAP), wherein
the control unit determines a typical expiration time over n breaths,
the control unit lowers the pressure from the IPAP to the EPAP taking into account the typical expiration time in such a way that the pressure drop to the EPAP is already reached to the extent of at least 85% after a proportion of the typical expiration time in the range of 40-60% of the typical expiration time, the EPAP after completion of the pressure drop being predefined until the end of the typical expiration time.

### CROSS-REFERENCE TO RELATED APPLICATIONS

The present application claims priority under 35 U.S.C. § 119 of German Patent Application No. 102021120415.1, filed Aug. 5, 2021, the entire disclosure of which is expressly incorporated by reference herein.

### BACKGROUND OF THE INVENTION

### 1. Field of the Invention

The invention relates to ventilation devices which predefine at least one adjustable expiratory positive airway pressure (EPAP) and also an adjustable inspiratory positive airway pressure (IPAP). With these devices, breaths are triggered by the patient, for example. The EPAP can correspond to a PEEP. PEEP signifies positive end-expiratory pressure and denotes the positive pressure that remains in the lungs at the end of exhalation. The invention relates to a device which lowers the IPAP to the EPAP in a targeted manner and thus controls the duration and the profile of the exhalation phase.

### 2. Discussion of Background Information

Devices of this kind can be used in particular in connection with ventilators, which are used for patients with COPD. Typical symptoms are emphysema and chronic obstructive bronchitis. These diseases can cause small airways to collapse during expiration in the context of ventilation. Accordingly, the expiration is incomplete and there is an increase in the intrapulmonary pressure (also intrinsic PEEP). The impeded exhalation leads to a pressure increase in the thorax. This results in less ventilation of the lungs.

US 2006/011195 A1, the entire disclosure of which is incorporated by reference herein, discloses a base pressure initially being predefined, which is then lowered to an exhalation level and then raised to a peak value. Thereupon, the pressure is lowered again to the base pressure. US 2006/011195 A1 discloses three different pressure levels for the ventilation. The pressure decreases take place passively.

WO 99/45989, the entire disclosure of which is incorporated by reference herein, discloses an exhalation assistance in which the pressure drops further below the expiratory base pressure (EPAP). Here, depending on the respiratory gas flow, an amplification factor is determined, and the exhalation assistance is multiplied by the amplification factor.

U.S. Pat. No. 3,961,627, the entire disclosure of which is incorporated by reference herein, discloses the automation of a pressure-controlled or volume-controlled ventilation. Four phases are distinguished, wherein phases III and IV serve for expiration, and wherein the duration of phase IV is never shorter than the duration of phase III, so as to avoid hyperdistension of the lungs (air trapping).

EP 2 542 286 B1, the entire disclosure of which is incorporated by reference herein, discloses a controlled pressure profile from the IPAP to the EPAP, where pressure in the exhalation phase is even increased intermittently.

EP 2 514 469 B1, the entire disclosure of which is incorporated by reference herein, discloses a controlled pressure drop from the IPAP to the PEEP (or EPAP) in three phases. In a first phase, the pressure drop is faster than in a second phase. After conclusion of the second phase, the pressure drop, in a third phase, is faster than in the second phase. The PEEP is reached only at the end of the exhalation time.

In view of the foregoing, it would be advantageous to have available a device which, by means of intelligent and fault-tolerant device control, supports sufficient expiration. It is ideally configured in such a way that the collapsible areas of the lungs remain opened, at least during a first part of the ramp time, and are thus partially emptied of air, and there is still enough time for the non-collapsed areas of the lungs to empty of air to the EPAP level.

### SUMMARY OF THE INVENTION

According to the invention, the pressure transition from one pressure level (IPAP or EPAP) to another is controlled on the basis of at least one characteristic time of an inspiration or expiration.

The present invention further provides a ventilation device which comprises a controllable respiratory gas source. The device further comprises a programmable control unit which is configured to perform the following steps:
determining the respiratory gas flow, the respiratory gas flow being used to determine whether an inspiration or an expiration is provided,
regulating the pressure for an inspiration (IPAP) and an expiration (EPAP), wherein
the control unit takes into account a typical expiration time,
the control unit lowers the pressure from the IPAP to the EPAP, taking into account the typical expiration time in such a way that the pressure drop to the EPAP is already reached to the extent of at least about 85% after a proportion of the typical expiration time in the range of about 40-60% of the typical expiration time, the EPAP after completion of the pressure drop being predefined until the end of the typical expiration time.

The control unit can determine a typical expiration time over n breaths and take into account the expiration time thus determined. The control unit can also take into account a fixedly predefined expiration time, which is predefined either explicitly or implicitly via the specification of an inspiration time and of a respiratory frequency.

It is preferable and advantageous that the pressure is lowered from the IPAP to the EPAP in the form of an adaptive pressure ramp, wherein the pressure reaches the EPAP after about 40-60%, preferably after about 50%, of the typical expiration time.

In particular, the device is suitably configured and designed such that the pressure drop to the EPAP is already reached to the extent of about 90% after a proportion or fraction of the typical expiration time.

The device is also suitably configured and designed to predefine an adaptive pressure ramp in which the pressure reaches the EPAP after about 40-60%, preferably after about 50%, of the typical expiration time.

The drop to the EPAP can be reached to the extent of at least about 80%, preferably to the extent of about 90% and particularly preferably to the extent of about 100%, after about 40-60%, preferably after about 50%, of the typical expiration time.

It is also possible that the EPAP is reached after a proportion of the typical expiration time, which is about 50%.

The device comprises at least, for example, a flow sensor and/or a pressure sensor or equivalent components.

In an advantageous further development, the device is designed and configured such that, after detection of an exhalation phase, the pressure is lowered from the IPAP to the EPAP such that the pressure is applied as a dynamically regulated counterpressure against the respiratory gas flow of the expiration, as a result of which the lower airways in COPD patients with expiratory flow limitation remain supported for approximately half of the typical expiration time and do not collapse.

In an advantageous development, the device is suitably configured and designed such that the pressure is reduced from the IPAP to the EPAP as a non-linear drop with asymptotic approximation to the EPAP.

In another development, the device is suitably configured and designed such that the pressure is reduced from the IPAP to the EPAP such that 90% of the pressure drop is reached after approximately half of the expiration time.

In an advantageous embodiment, the device is suitably configured and designed such that the pressure is regulated only at the start of the expiration and, after the EPAP is reached, is left at this level.

Furthermore, the device is suitably configured and designed such that the control unit can detect an increase of the expiratory resistance, for example via an early flattening of the expiratory flow curve or a resistance measurement by means of forced oscillation technology, the pressure drop from the IPAP to the EPAP thus taking into account the expiratory resistance, for example by means of the ramp of the pressure drop being set flatter.

The device is for example further configured and designed such that the control unit detects an increased residual flow at the end of the expiration, for example by comparing the current flow values with the flow values of preceding expirations, the pressure drop from the IPAP to the EPAP taking into account the increased residual flow at the end of the expiration, for example by means of the ramp of the pressure drop being set steeper.

Alternatively or in addition, the device is configured and designed such that control unit varies the steepness of the pressure drop, for example from breath to breath, and determines at which steepness of the pressure drop the best emptying of air from the lungs takes place. The control unit determines this for example by calculating the expiratory tidal volume or measuring the residual flow left at the end of the expiration. This learning process can be regularly repeated during the ventilation.

The device is preferably suitably configured and designed such that the control unit changes the steepness of the pressure drop, for example based on how great a percentage of the expiration time the EPAP is intended to reach, according to a specification that is input via a data interface. The specification can be made by a user or automatically in the context of a remote control of the device via a network or the cloud. The specification can also be made by a user or automatically in the context of a control or input via an operating unit, on the basis of respiratory flow curves or further sensor signals or on the basis of the feedback from the patient. The specification can be effected manually or automatically. The specification can take account of at least one of the following parameters: age, size, disease, therapy goal, lung volume, lung compliance, lung resistance, respiratory effort, shortness of breath.

In particular, the device is suitably configured and designed such that the control unit controls a change in the steepness of the pressure drop on the basis of sensor signals, for example from effort belts, diaphragm EMG, esophageal pressure probes, body plethysmography or electrical impedance tomography. The sensor signals represent a measure of the filling of the lungs at the end of the inspiration and/or expiration and/or a measure of the change in the filling of the lungs in the course of the inspiration and/or expiration. This measure is used by the control unit to change the steepness of the pressure drop. In particular, the control unit also changes the steepness of the pressure drop on the basis of the change of the measure over time, or on the basis of this change over time corresponding to the change over time of the steepness of the pressure drop.

According to the invention, the device is also suitably configured and designed such that the control unit changes the steepness of the pressure drop when the pressure assistance is adapted, i.e. the difference between IPAP and EPAP is changed. The pressure assistance can be adapted either manually or automatically, for example in anticyclic servo-ventilation or in target volume control. In the case of increasing pressure assistance, the steepness of the pressure drop is typically reduced, such that the EPAP is reached only at a later time in the expiration. In this way, part of the more strongly falling therapeutic pressure is compensated, and the airways are thus more strongly supported against collapse.

The device can also be suitably configured and designed such that the control unit changes the steepness of the pressure drop when the EPAP is adapted. The steepness is preferably increased when the EPAP is increased, since stronger support of the lower airways is achieved in any case by the higher EPAP.

The device is preferably also suitably configured and designed such that
the control unit determines a typical inspiration time over n breaths,
the control unit raises the pressure from the EPAP to the IPAP taking into account the characteristic inspiration time, such that the IPAP is reached after a proportion of the typical inspiration time lying in the range of about 20-40%.

The device is furthermore configured and designed, for example, such that the control unit lowers the pressure from the EPAP to the IPAP taking into account the typical inspiration time, in such a way that the IPAP is reached after a proportion of the typical inspiration time lying in the region of 30%.

It is possible and preferable that the control unit raises the pressure from the EPAP to the IPAP in such a way that the result is a non-linear rise with asymptotic approximation to the IPAP.

It is also possible and preferable that the control unit raises the pressure from the EPAP to the IPAP in such a way that approximately 90% of the rise is reached after 30% of the inspiration time.

The device can be suitably configured and designed such that the pressure change (EPAP to IPAP or IPAP to EPAP) can be specified with a fixed duration or speed or step associated therewith.

Preferably, the device is suitably configured and designed such that the control unit determines the typical expiration time and/or typical inspiration time over at least (n) three breaths or preferably (n) 10 breaths, wherein a breath comprises an inspiration and an expiration. Such numbers of breaths have proven particularly reliable for the identification of a typical expiration time and/or typical inspiration time.

In addition, the device can also be suitably configured and designed such that the respiratory volume of the inspiration (AI) is determined, and the duration of the expiration is adapted when the respiratory volume of the expiration (AE) reaches the value of the respiratory volume of the inspiration (AI).

The invention relates primarily to ventilation devices such as APAP devices, bilevel devices, servo-ventilation devices, devices for home ventilation and for intensive care ventilation, and also emergency ventilators. The invention can be used with a leakage hose, a single-hose valve system or a double-hose system, in combination with the abovementioned ventilation devices.

The invention can be used in ventilation devices which permit spontaneous and/or mandatory ventilation. Spontaneous ventilation is a form of assistive ventilation in which the patients breathe by themselves. The patients control the respiratory frequency, and the ventilator assists the inhalation and/or exhalation by means of a pre-set pressure. The ventilation is brought about by a so-called trigger. The patients themselves generate a respiratory gas flow or pressure at the start of inhalation, which is recognized by the ventilator. If the respiratory gas flow or pressure generated by the patients exceeds the pre-set threshold, the device switches to the pressure for the inhalation and/or exhalation.

In mandatory ventilation, the device specifies the sequence of inhalation and exhalation. Here, the time of the exhalation is thus predefined (and is not determined as the typical expiration time).

According to the invention, in the case of spontaneous ventilation too, the exhalation can be ended earlier by the patient trigger, at least for one or a few breaths, such that the EPAP is possibly not yet reached or the typical expiration time is not yet reached.

Within the meaning of the invention, all of the described embodiments can be combined with one another.

It will be noted that the features individually presented in the claims can be combined with one another in any desired, technically meaningful way and show further refinements of the invention. The description additionally characterizes and specifies the invention in particular in conjunction with the drawings.

It will also be noted that an “and/or” conjunction used herein between two features, and linking them to each other, is always to be interpreted as meaning that in a first embodiment of the subject matter according to the invention only the first feature may be present, in a second embodiment only the second feature may be present, and in a third embodiment both the first and the second feature may be present.

### BRIEF DESCRIPTION OF THE DRAWINGS

Further advantages and features of the present invention will become clear from the description of the illustrative embodiments, which are explained below with reference to the accompanying drawings. In the drawings,

FIG. 1 shows a device according to the invention with a breathing mask as a patient interface;

FIG. 2A shows the respiratory gas flow that has been recorded by sensors of a device of the invention;

FIG. 2B shows how a device of the prior art predefines the IPAP and the EPAP in the alternation of inspiration and expiration; and

FIG. 2C shows how a device of the invention predefines the IPAP and the EPAP in the alternation of inspiration and expiration;

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the 5 present invention. In this regard, no attempt is made to show details of the present invention in more detail than is necessary for the fundamental understanding of the present invention, the description in combination with the drawings making apparent to those of skill in the art how the several forms of the present invention may be embodied in practice.

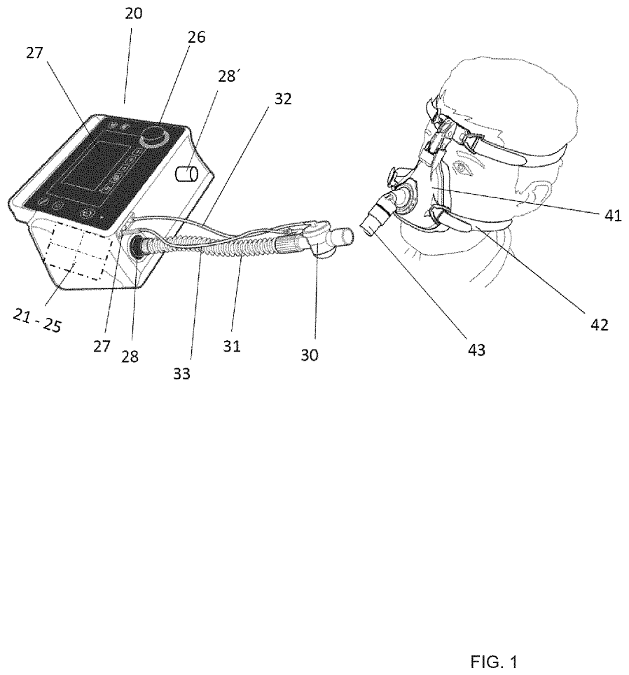

FIG. 1 shows the device 20 according to the invention with a breathing mask 41 as a patient interface. The mask is fastened on the head with straps 42. The mask can be connected to the hose via a connector 43.

The device 20 comprises a respiratory gas source 21, a control unit 22, a reservoir 25, a pressure sensor device 24 and/or a flow sensor device 23, a respiratory gas hose 31 and a patient interface, which is here designed as a breathing mask 41. The device additionally has an operating unit 26 and a display 27. The device has at least one connector 28 for the respiratory gas hose 31. A connector 28 is configured for the attachment of the one respiratory gas hose 31 in the form of a single-hose valve system 31. A leakage hose can also be attached to this connector.

Moreover, the inspiratory branch of a double-hose system can be attached to this connector 28. The other connector 28′ serves for the attachment of the expiratory branch of the double-hose system.

The device can be used with a leakage hose, a single-hose valve system or a double-hose system. In the leakage hose, the CO2-containing exhaled air is continuously flushed out via an exhalation system. In the single-hose valve system and in the double-hose system, the exhalation by the patient is controlled via a valve 30.

In the double-hose system, the valve 30 is arranged in the device. The exhaled air is conveyed via a subsidiary hose to the expiratory input connector 28′ and from there is discharged via the valve 30 into the atmosphere. The valve for this purpose opens with each expiration. The valve is closed with each inspiration.

A pressure measurement hose 32 registers the pressure in the hose system. The control pressure for the valve 30 comes from the device via a pressure hose 35.

In the single-hose valve system, the valve 30 is arranged in or on the hose 31.

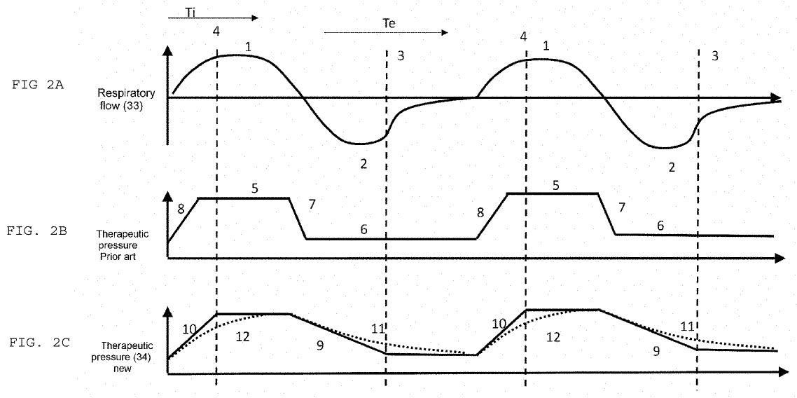

FIG. 2A shows the respiratory gas flow 33 that has been recorded by sensors 23 of the device 20. The inspiration 1 by the patient and the expiration 2 by the patient are seen from the flow signal 33. Inspiration and expiration alternate with each other in the breathing rhythm of the patient. From the flow 33, the control unit can detect the change between inspiration and expiration and can accordingly alter the pressure specification 34. From the flow, the control unit can detect the change between inspiration and expiration and determine the durations of inspiration (Ti) and expiration (Te).

FIG. 2B and FIG. 2C show that the device predefines the IPAP 5 and the EPAP 6 in the alternation of inspiration and expiration.

It will be seen from FIG. 2B that, in devices according to the prior art, the change from the IPAP to the EPAP takes place for the most part over a ramp-shaped pressure drop 7. The EPAP is reached shortly after the change of respiration phase. The change from the EPAP to the IPAP also takes place for the most part over a ramp-shaped pressure rise 8. The IPAP is then also reached quickly. The ramps 7, 8 can each typically be set or predefined with a fixed duration or speed.

FIG. 2C shows the innovation according to the invention. The device 20 controls 22 the pressure drop from the IPAP 5 to the EPAP 6, such that the EPAP is reached after half of the typical expiration time 3, or 90% of the pressure drop is reached after half of the typical expiration time 3. For this purpose, the ventilation device 20 has a controllable respiratory gas source (21) and a programmable control unit (22).

The programmable control unit (22) is configured to perform the following steps:
determining the respiratory gas flow (33), the respiratory gas flow (33) being used to determine whether an inspiration or an expiration is provided,
regulating the pressure for an inspiration (IPAP) and an expiration (EPAP), wherein
the control unit (22) determines a typical expiration time 3 over n breaths,
the control unit (22) lowers the pressure from the IPAP (5) to the EPAP (6) taking into account the typical expiration time (3), in such a way that the pressure drop to the EPAP (6) is already reached to the extent of least about 90% after a proportion or fraction of the typical expiration time (3).

In the example of the adaptive pressure ramp 9, the pressure reaches the EPAP after about 40-60%, preferably after about 50%, of the typical expiration time 3.

In the example of the non-linear drop 11 with asymptotic approximation to the EPAP, the drop to the EPAP reaches at least about 80%, preferably about 90%, after 40-60%, preferably after about 50%, of the typical expiration time 3.

The shift is effected, for example, by the control unit determining the mean expiration time 3 using a weighted average filter, which takes account of about the last 10 breaths. The pressure is lowered from IPAP to EPAP at a suitable speed, such that the EPAP is reached after about 50% of the average expiration time. There is therefore still enough time for the non-collapsed areas of the lungs to empty of air to the EPAP level. The collapsible areas remain opened at least during part of the ramp time and are thus partially emptied of air.

The setpoint expiration times or inspiration times, to which the % target values of the EPAP/IPAP attainment and thus the ramp gradients relate, can be derived from the spontaneous breathing pattern of the patient.

The setpoint expiration times or inspiration times, to which the % target values of the EPAP/IPAP attainment and thus the ramp gradients relate, can alternatively or additionally also be predefined as ideal inspiration or expiration times.

The specification can be made manually or performed automatically on the basis of at least one of the parameters: age, size, disease, therapy goal, lung volume, lung compliance, lung resistance, respiratory effort, shortness of breath.

FIG. 2C also shows the innovation according to the invention for the pressure rise. The device 20 controls 22 the pressure rise to the IPAP 5, such that the IPAP is reached after 20-40%, preferably after 30%, of the typical inspiration time.

In the example of the adaptive pressure ramp, the pressure reaches the IPAP after 30% of the typical inspiration time.

The shift is effected, for example, by the control unit determining the typical inspiration time 4 using a weighted average filter, which takes account of about the last 10 breaths. The pressure is raised from EPAP to IPAP at a suitable speed, such that the IPAP is reached after about 30% of the typical inspiration time.

In the example of the non-linear rise 12 with asymptotic approximation to the IPAP, the rise at least preferably to the extent of about 90% after 40-60%, preferably after 50%, of the typical expiration time.

In the example of the non-linear drop 11 with asymptotic approximation to the EPAP, the drop to the EPAP reaches at least about 80%, preferably about 90%, after about 20-40%, preferably after about 30%, of the typical inspiration time. FIG. 2C shows
1 inspiration by the patient
2 expiration by the patient
3 50% of the typical expiration time (Te)
4 30% of the typical inspiration time (Ti)
5 IPAP level (inspiratory pressure)
6 EPAP level (expiratory pressure)
7 pressure ramp IPAP>EPAP, typically with fixed duration or speed or step associated therewith
8 pressure ramp EPAP>IPAP, typically with fixed duration or speed or step associated therewith
9 adaptive pressure ramp, reaches the EPAP after about 50% of the typical expiration time
10 adaptive pressure ramp, reaches the IPAP after about 30% of the typical inspiration time
11 alternative embodiment of the ramp; no linear drop, instead a non-linear drop with asymptotic approximation to the EPAP; characteristic value: e.g. about 90% of the drop reached after about 50% of the expiration time
12 alternative embodiment of the ramp; no linear rise, instead a non-linear rise with asymptotic approximation to the IPAP; characteristic value: e.g. about 90% of the rise reached after about 30% of the inspiration time

According to the invention, the control of the expiration ramp is as flat as possible, so that the lower airways in COPD patients with expiratory flow limitation remain supported for as long as possible and do not collapse. In this way, air is better removed from the lungs and there is a lower intrinsic PEEP. The patient can breathe more easily, since the lungs remain less distended, and can release the trigger. After the ventilator has been switched off, breathing is found to be easier.

The shift is effected, for example, by the mean expiration time being determined by a weighted average filter, which takes account of ca. the last 10 breaths. The pressure is lowered from IPAP to EPAP at a suitable speed, such that the EPAP is reached after about 50% of the average expiration time. There is therefore still enough time for the non-collapsed areas of the lungs to empty of air to the EPAP level. The collapsible areas remain opened at least during part of the ramp time and are thus partially emptied of air.

To sum up, the present invention provides:
1. A ventilation device which comprises a controllable respiratory gas source and a programmable control unit, the programmable control unit being configured to:
   determine a respiratory gas flow, the respiratory gas flow being used to determine whether an inspiration or an expiration is present,
   regulate a pressure for an inspiration (IPAP) and an expiration (EPAP), wherein
   the control unit takes into account a typical expiration time,
   the control unit lowering the pressure from the IPAP to the EPAP taking into account a typical expiration time in such a way that a pressure drop to the EPAP is already reached to an extent of at least 85% after a proportion of the typical expiration time in a range of 40-60% of the typical expiration time, the EPAP after completion of the pressure drop being predefined until an end of the typical expiration time.
2. The device of item 1, wherein the control unit determines a typical expiration time over n breaths and takes into account the expiration time thus determined, or the control unit takes into account a fixedly predefined expiration time, which is predefined either explicitly or implicitly via the specification of an inspiration time and of a respiratory frequency.
3. The device of at least one of the preceding items, wherein the pressure is lowered from the IPAP to the EPAP in the form of an adaptive pressure ramp, the pressure reaching the EPAP after 40-60%, preferably after 50%, of the typical expiration time.
4. The device of at least one of the preceding items, wherein the EPAP is reached after a proportion of the typical expiration time, which is 50%.
5. The device of at least one of the preceding items, wherein, after detection of an exhalation phase, the pressure is lowered from the IPAP to the EPAP such that the pressure is applied as a dynamically regulated counterpressure against the respiratory gas flow of the expiration, as a result of which the lower airways in COPD patients with expiratory flow limitation remain supported for approximately half of the typical expiration time and do not collapse.
6. The device of at least one of the preceding items, wherein the pressure is reduced from the IPAP to the EPAP as a non-linear drop with asymptotic approximation to the EPAP.
7. The device of at least one of the preceding items, wherein the pressure is reduced from the IPAP to the EPAP such that 90% of the pressure drop is reached after approximately half of the expiration time.
8. The device of at least one of the preceding items, wherein the pressure is regulated only at the start of the expiration and, after the EPAP is reached, is left at this level.
9. The device of at least one of the preceding items, wherein the control unit is configured and designed to detect an increase of the expiratory resistance and to perform the pressure drop from the IPAP to the EPAP taking into account the expiratory resistance, for example by means of the ramp of the pressure drop being set flatter.
10. The device of at least one of the preceding items, wherein the control unit is configured and designed to detect an increased residual flow at the end of the expiration and to perform the pressure drop from the IPAP to the EPAP taking into account the increased residual flow at the end of the expiration, for example by means of the ramp of the pressure drop being set steeper.
11. The device of at least one of the preceding items, wherein the control unit is configured and designed to vary the steepness of the pressure drop, for example from breath to breath, and to determine at which steepness of the pressure drop the best emptying of air from the lungs takes place.
12. The device of at least one of the preceding items, wherein the control unit is configured and designed to change a steepness of the pressure drop, for example based on how great the percentage of the expiration time the EPAP is intended to reach, according to the specification that is input via a data interface.
13. The device of at least one of the preceding items, wherein the control unit is configured and designed to control a change in the steepness of the pressure drop on the basis of sensor signals, for example from effort belts, diaphragm EMG, esophageal pressure probes, body plethysmography or electrical impedance tomography.
14. The device of at least one of the preceding items, wherein the control unit is configured and designed to change the steepness of the pressure drop when the pressure assistance is adapted, i.e., the difference between IPAP and EPAP is changed.
15. The device of at least one of the preceding items, wherein the control unit is configured and designed to change the steepness of the pressure drop when the EPAP is adapted.
16. The device of at least one of the preceding items, wherein
   the control unit determines a typical inspiration time over n breaths,
   the control unit raises the pressure from the EPAP to the IPAP taking into account the characteristic inspiration time, such that the IPAP is reached after a proportion of the typical inspiration time in a range of 20-40%.
17. The device of at least one of the preceding items, wherein the control unit lowers the pressure from the EPAP to the IPAP taking into account the typical inspiration time, in such a way that the IPAP is reached after a proportion of the typical inspiration time in a region of 30%.
18. The device of at least one of the preceding items, wherein the control unit raises the pressure from the EPAP to the IPAP in such a way that the result is a non-linear rise with asymptotic approximation to the IPAP.
19. The device of at least one of the preceding items, wherein the control unit raises the pressure from the EPAP to the IPAP in such a way that approximately 90% of the rise is reached after 30% of the inspiration time.
20. The device of at least one of the preceding items, wherein the pressure change (EPAP to IPAP or IPAP to EPAP) can be specified with a fixed duration or speed or step associated therewith.
21. The device of at least one of the preceding items, wherein the control unit determines the typical expiration time and/or typical inspiration time over at least three breaths or preferably 10 breaths, wherein a breath comprises an inspiration and an expiration.
22. The device of at least one of the preceding items, wherein a respiratory volume of the inspiration (AI) is determined, and the duration of the expiration is adapted when the respiratory volume of the expiration (AE) reaches the value of the respiratory volume of the inspiration (AI).

What is claimed is:

1. A ventilation device, wherein the device comprises a controllable respiratory gas source and a programmable control unit, the programmable control unit being configured to:
determine a respiratory gas flow, the respiratory gas flow being used to determine whether an inspiration or an expiration is present,
regulate a pressure for an inspiration (IPAP) and an expiration (EPAP), wherein
the control unit takes into account a typical expiration time,
the control unit lowers the pressure from the IPAP to the EPAP taking into account a typical expiration time in such a way that a pressure drop to the EPAP is already reached to an extent of at least 85% after a proportion of the typical expiration time in a range of 40-60% of the typical expiration time, the EPAP after completion of the pressure drop being predefined until an end of the typical expiration time.

2. The device of claim 1, wherein the control unit determines a typical expiration time over n breaths and takes into account the expiration time thus determined, or the control unit takes into account a fixedly predefined expiration time, which is predefined either explicitly or implicitly via a specification of an inspiration time and of a respiratory frequency.

3. The device of claim 1, wherein the pressure is lowered from the IPAP to the EPAP in the form of an adaptive pressure ramp, the pressure reaching the EPAP after 40-60% of the typical expiration time.

4. The device of claim 1, wherein the EPAP is reached after a proportion of the typical expiration time, which is 50%.

5. The device of claim 1, wherein, after detection of an exhalation phase, the pressure is lowered from the IPAP to the EPAP such that the pressure is applied as a dynamically regulated counterpressure against the respiratory gas flow of the expiration, as a result of which lower airways in COPD patients with expiratory flow limitation remain supported for approximately half of the typical expiration time and do not collapse.

6. The device of claim 1, wherein the pressure is reduced from the IPAP to the EPAP as a non-linear drop with asymptotic approximation to the EPAP.

7. The device of claim 1, wherein the pressure is reduced from the IPAP to the EPAP such that 90% of the pressure drop is reached after approximately half of the expiration time.

8. The device of claim 1, wherein the pressure is regulated only at a start of the expiration and, after the EPAP is reached, is left at this level.

9. The device of claim 1, wherein the control unit is configured and designed to detect an increase of an expiratory resistance and to perform the pressure drop from the IPAP to the EPAP taking into account the expiratory resistance.

10. The device of claim 1, wherein the control unit is configured and designed to detect an increased residual flow at an end of the expiration and to perform the pressure drop from the IPAP to the EPAP taking into account the increased residual flow at the end of the expiration.

11. The device of claim 1, wherein the control unit is configured and designed to vary a steepness of the pressure drop and to determine at which steepness of the pressure drop a best emptying of air from lungs takes place.

12. The device of claim 1, wherein the control unit is configured and designed to change a steepness of the pressure drop according to a specification that is input via a data interface.

13. The device of claim 1, wherein the control unit is configured and designed to control a change in a steepness of the pressure drop on the basis of sensor signals.

14. The device of claim 1, wherein the control unit is configured and designed to change a steepness of the pressure drop when a pressure assistance is adapted, i.e., a difference between IPAP and EPAP is changed.

15. The device of claim 1, wherein the control unit is configured and designed to change a steepness of the pressure drop when the EPAP is adapted.

16. The device of claim 1, wherein
the control unit determines a typical inspiration time over n breaths, and
the control unit raises the pressure from the EPAP to the IPAP taking into account the characteristic inspiration time, such that the IPAP is reached after a proportion of the typical inspiration time in a range of 20-40%.

17. The device of claim 1, wherein the control unit lowers the pressure from the EPAP to the IPAP taking into account a typical inspiration time in such a way that the IPAP is reached after a proportion of the typical inspiration time in a region of 30%.

18. The device of claim 1, wherein the control unit raises the pressure from the EPAP to the IPAP in such a way that a result is a non-linear rise with asymptotic approximation to the IPAP.

19. The device of claim 1, wherein the control unit raises the pressure from the EPAP to the IPAP in such a way that approximately 90% of a rise is reached after 30% of the inspiration time.

20. The device of claim 1, wherein the pressure change (EPAP to IPAP or IPAP to EPAP) can be specified with a fixed duration or speed or step associated therewith.
